(19) United States
(12) Patent Application Publication
(10) Pub. No.: US 2012/0222349 A1
(43) Pub. Date: Sep. 6, 2012

(54) ONE-STEP HYDRODEOXYGENATION AND REFORMATION OF ALDITOLS

(75) Inventors: Matthew Jarrod Truitt, Bartlesville, OK (US); Bradley Martin Taylor, Tulsa, OK (US)

(73) Assignee: ConocoPhillips Company, Houston, TX (US)

(21) Appl. No.: 13/410,800

(22) Filed: Mar. 2, 2012

Related U.S. Application Data
(60) Provisional application No. 61/448,862, filed on Mar. 3, 2011.

(51) Int. Cl. C10L 1/00; C07D 309/00

(52) U.S. Cl. 44/349; 549/356

(57) ABSTRACT

This application provides an efficient method for fuel processing, comprising providing a fluid hydrocarbon fuel feedstock comprising an oxygenate; contacting the feedstock with a catalyst comprising Pt loaded with WO₃ and ZrO₂ in he presence of hydrogen; reacting the feedstock, hydrogen and catalyst at a temperature and a pressure for a period of time sufficient to allow hydrodeoxygenation and hydrocarbon isomerization of one or more oxygenates in the feedstock.

### CROSS-REFERENCE TO RELATED APPLICATIONS

This application is a non-provisional application which claims the benefit of and priority to U.S. Provisional Application Ser. No. 61/448,862 filed Mar. 3, 2011, entitled “One-Step Hydrogenation and Reformation of Alditols,” which is hereby incorporated by reference in its entirety.

### FIELD OF THE INVENTION

The invention relates to reforming and upgrading a hydrocarbon fuel feedstock, in particular to a one-step hydrodeoxygenation and hydrocarbon isomerization of a hydrocarbon fuel containing oxygenates.

### BACKGROUND OF THE INVENTION

Biomass is of increasing interest as a source of renewable fuels, fine chemicals and energy. Typically, biomass has been used to make ethanol, which is used as a fuel. However, ethanol is not an ideal fuel, suffering from problems such as high hygroscopicity, high vapor pressure and low energy density. These qualities make ethanol incompatible with the current facilities used in the storage, distribution and use of liquid transportation fuels.

Biomass can also be used to make hydrogen. However, although of some interest, hydrogen also requires a different distribution system that current fuels, and requires different technologies for use as well. Thus, we are a long way yet from moving to a hydrogen economy.

Another common fuel derived from biomass is biodiesel, and biodiesel has the advantage of being much more compatible with existing infrastructure. Biodiesel refers to a vegetable oil- or animal fat-based diesel fuel consisting of long-chain alkyl (methyl, propyl or ethyl) esters. Biodiesel is typically made by chemically reacting lipids (e.g., vegetable oil, animal fat (tallow)) with an alcohol. However, although compatible with existing fuel distribution infrastructure, and important fuel in some European and developing countries, the energy efficiency of biodiesel is still debated. Further, biodiesel tends to require larges tracts of productive land, and thus competes with food crops for this limited resource.

Cellulosic energy sources allows use of the entire plant, and also allows access to waste materials. However, most efforts in this area to date have been directed to producing ethanol or hydrogen from cellulosic raw materials, and the disadvantages of these are already discussed. It would be advantageous if, instead, a fuel more comparable to conventional gasoline or diesel could be produced from cellulosic resources such as native grasses and waste. One of the products that can be obtained from cellulosic biomass are polyols, and thus interest in polyol products is increasing. However, polyols need to be further improved to make better fuels.

Using typical sulfided hydrodesulfurization catalysts and hydrogen gas, oxygen can be removed from a polyol or oxygenate present in a fuel feedstock. The oxygenate is converted into its respective alkane, but the alkanes produced from this process have a very poor octane rating, and must be reformed to make them suitable for use in fuel. That is, they must be isomerized into more branched alkanes in order to provide a suitable combustion. Furthermore, because the catalysts are sulfided, they can release sulfur into the product stream, resulting in unwanted material that can damage catalysts used in other refinery processes.

Kurosaka et al. (“Production of 1,3-propanediol by hydrogenolysis of glycerol catalyzed by Pt/WO₃/ZrO₂”, Catalysis Communications, 2008, 9(6): 1360-1363) describe glycerol hydrogenolysis catalyzed by Pt/WO₃—ZrO₂ to give 1,3-propanediol (propylene glycol) in yields up to 24%. Gong et al. (“Solvent Effect on Selective Dehydroxylation of Glycerol to 1,3-Propanediol over Pt/WO₃/ZrO₂”, Chinese Journal of Catalysis, 2009, 30(12): 1189-1191) adds that binary solvent containing a protic component displays a synergistic solvent effect in selective dehydroxylation of glycerol to 1,3-propanediol. However, because glycerol contains only three carbon atoms, it cannot be isomerized to have a more highly branched alkyl backbone.

U.S. Pat. No. 4,338,472 and U.S. Pat. No. 4,366,332 describe a particulate, stabilized, nickel catalyst containing 50-60 weight % porous nickel on silica used to produce glycerol from alditol solution through hydrogenolysis.

US2008216391 describes a method of deoxygenating an alditol using a metal oxide catalyst loaded with a noble metal.

US20090255171 describes C₄-C₆ ketones and secondary alcohols derived from sorbitol using a Pt—Re/C catalyst, forming C—C bonds through aldol condensation over a basic catalyst to produce C₈-C₁₂ compounds, and then yielding C₈-C₁₂ alkanes through hydrodeoxygenation over Pt/Nb₂O₅ at 548 K.

Busto et al. (“Pt—Pd/WO₃—ZrO₂ catalysts for isomerization-cracking of long paraffins”, Applied Catalysis A: General, 2008, 347(2): 117-125) describe Pt—Pd/WO₃—ZrO₂-catalyzed isomerization and cracking of long-chain alkanes, particularly n-decane, and investigate the relationship between catalytic activity, tungsten content, and calcination temperature.

Hattori et al. (“Participation of the Protonic Acid Sites Originating from Molecular Hydrogen in Alkane Skeletal Isomerization Catalyzed by Pt/WO₃—ZrO₂”, Proceedings of 13^{th} Saudi-Japanese Catalyst Symposium, December 2003, 124-133) describe Pt/WO₃—ZrO₂ catalysis of alkane skeletal isomerization, particularly in the presence of hydrogen.

Hong et al. (“Hydrodeoxygenation and coupling of aqueous phenolics over bifunctional zeolite-supported metal catalysts.” Chem Commun, 2010, 46: 1038-1040) (2010) describe Pt-supported on zeolite used as a bifunctional catalyst for phenol hydrodeoxygenation in a fixed-bed configuration at elevated hydrogen pressures, leading to hydrogenation-hydrogenolysis ring-coupling reactions which produced hydrocarbons.

Khurshid and Al-Khattaf (“n-Heptane isomerization over Pt/WO₃—ZrO₂: A kinetic study”, Applied Catalysis A: General, 2009, 368(1-2): 56-64) describe kinetics and the effect of reaction conditions on reactivity of n-heptane isomerization over Pt-loaded WO₃—ZrO₂ using a fixed-bed reactor under hydrogen. Cracked product mainly comprises di-branched isomers of heptane, especially 2,3-dimethylpentane, with several minor products, including 3-methyhexane, propane, and isobutane.

Lukinskas et al. (“Role of promoters on tungstated zironia catalysts”, Topics in Catalysis, 2003, 23(1-4): 163) describe isomerization of straight-chain alkanes catalyzed by tungstated zironia promoted by Pt or by Pt and Fe, and explore calcination temperatures used in catalyst formation and Pt particle size.

Song et al. (“n-Hexane Isomerization by Pt/WO₃—ZrO₂ Using Hydrothermally Synthesized Hydrous Zirconia as Support”, Chinese J. of Catal., 2008, 29(12): 1196-1198) describe Pt/WO₃—ZrO₂-catalyzed isomerization of n-hexane to form branched alkanes.

Triwahyono et al. (“Molecular Hydrogen Originated Protonic Acid Site on Pt/WO₃—ZrO₂”, Journal of Natural Gas Chemistry, 2007, 16(3): 252-257) describe a mechanism for hydrogen adsorption to Pt/WO₃—ZrO₂, and use the proposed mechanism to explain why Pt/WO₃—ZrO₂ is selective for alkyl isomerization over cracking as compared to Pt/SO₄—ZrO₂.

Yori et al. “n-Butane isomerization of Pt/WO₃—ZrO₂: effect of the Pt incorporation”, Applied Catalysis A: General, 1999, 181(1): 5-14) describe the catalysis of n-butane isomerization using Pt/WO₃—ZrO₂ in the presence of hydrogen gas.

However, there is still lacking an efficient process to hydrodeoxygenate and isomerize a hydrocarbon feedstock comprising an oxygenate, such as an alditol. Such methods, if available, would be an important improvement in our ability to utilize renewable resources and move towards a green economy.

### SUMMARY OF THE INVENTION

We have discovered herein that a non-sulfided catalyst, Pt-loaded on WO₃ and ZrO₂ (Pt/WO₃—ZrO₂), can both simultaneously upgrade and reform a paraffin stream. That is, Pt/WO₃—ZrO₂ can hydrodeoxygenate oxygenate in a fuel feedstock to produce the corresponding n-alkanes, and isomerize the n-alkanes to branched alkanes and/or cycloalkanes in the same process step, producing a high-octane blendstock. The method is particularly useful when the oxygenate is an alditol-found in a biomass fuel. Hydrodeoxygenation can be complete, producing saturated alkanes, or partial, resulting in a complex mixture of intermediately deoxygenated products, such as hexanone, hexanol, hexanoic acid, and cyclic ethers. With further processing, these products can be fully deoxygenated.

This method has several key advantages. It does not require a Claus unit because no hydrogen sulfide gas (H₂S) is produced. It also does not require continuous injection of a sulfiding agent to maintain catalytic activity. Most of all, this method provides a one-step hydrodeoxygenation and isomeration process. This simpler and cheaper method for reforming and upgrading biomass-derived oxygenates and eliminates the separate upgrading and reforming steps typically needed to produce biofuels from biomass-derived oxygenates. Thus, this method saves time, money, effort and energy compared to previous methods.

In particular, this application provides a method for hydrocarbon processing, comprising: providing a fluid hydrocarbon feedstock comprising an oxygenate; contacting the feedstock with a catalyst comprising Pt-loaded WO₃/ZrO₂ in the presence of hydrogen; and reacting the feedstock, hydrogen and catalyst at a temperature and a pressure for a period of time sufficient to allow hydrodeoxygenation and hydrocarbon isomerization of said oxygenate. The fluid hydrocarbon feedstock can comprise an aqueous oxygenate stream and a fluid hydrocarbon stream. These two streams can be provided separately, sequentially or substantially simultaneously.

The ratio of WO₃ to ZrO₂ in the catalyst can be from 1:9 to 2:8. Before reaction, the catalyst can be formed at a calcination temperature of 400° C. to 800° C., for example 700° C., for 2 hours to 4 hours, for example 3 hours, so that tetragonoal zirconia content of the catalyst is increased. The weight percent of Pt in the catalyst can be 0.01 wt % to 10 wt % of the weight of the catalyst, for example 1 wt % to 5 wt % of the weight of the catalyst. The catalyst can be substantially free of sulfide. The catalyst can have an average particle size of 10 μm to 10 mm, such as 100 μm to 500 μm. The catalyst can also be porous.

The method can further comprise separating a product from the feedstock, hydrogen and catalyst. In preferred embodiment, the feedstock is biomass or derived from biomass. The oxygenate can comprise at least one alditol, and the alditol can be selected from the group consisting of erythritol, threitol, arabitol, xylitol, ribitol, sorbitol, iditol, dulcitol, and mannitol, and mixtures thereof. The temperature can be from 100° C. to 300° C., such as from 150° C. to 250° C. The pressure can be 800 psig to 2500 psig, and the period of time can be from 15 minutes to 5 hours.

In a particular embodiment, there is provided a method for hydrodeoxygenation and isomerization of an alditol, comprising: providing substantially simultaneously a fluid hydrocarbon stream and an aqueous stream comprising at least one alditol; contacting the streams with a catalyst comprising Pt-loaded WO₃/ZrO₂ in the presence of hydrogen; and reacting the streams, hydrogen and catalyst from 100° C. to 300° C. at 800 psig to 2500 psig for 15 minutes to 5 hours to allow hydrodeoxygenation and isomerization of the alditol.

### DESCRIPTION OF EMBODIMENTS OF THE INVENTION

The following abbreviations are used herein:

| | |
|---|---|
| DI | deionized water |
| HSD | hydrodesulfurization |
| WZ | tungstated zirconia |
| ZSM | zeolite sieve of molecular |
| SCCM | Standard Cubic Centimeters per Minute, |
| | a flow measurement term |
| PSIG | pound-force per square inch gauge |

“Alditol” refers to an acyclic polyol derived by reducing the aldehyde of an aldose or ketone of a ketose having at least 4 carbons, for example a tetrose, pentose or hexose. As such, alditol is a class of sugar alcohols. Examples of alditol include, but are not limited to erythritol, threitol, arabitol, xylitol, ribitol, sorbitol (glucitol), iditol, dulcitol (galactitol), and mannitol. As used herein, alditol does not include ethylene glycol or glycerol, which only have 2 and 3 carbons, respectively.

“Aldose” is a monosaccharide (a simple sugar) comprising one aldehyde group per molecule and having the chemical formula Cₙ(H₂O)ₙ, wherein n is an integer greater than or equal to 2. Examples of aldoses include, but are not limited to, dioses, such as glycoladehyde; trioses, such as glyceraldehyde; tertroses, such as erythrose and threose; pentoses, such as ribose, arabinose, xylose, and lyxose; and hexoses, such as allose, altrose, glucose, mannose, gulose, idose, galactose, and talose.

“Biomass” refers to any biological material from living, or recently living organisms, such as wood, paper, agricultural waste, consumer waste, wood and paper waste, cereal and grass crops, vegetable and tree oils, algae, and the like.

“Hydrocarbon fuel” refers to any fuel, including fossil fuels, biomass fuels, and the like. “Fossil fuel” refers to fuel formed from natural resources, such as anaerobic decomposition of organisms, for example phytoplankton, zooplankton, and plant matter. Fossil fuels can include coal, crude oil, and natural gas. “Biomass fuel” or “biofuel” refers to a fuel derived from biological material of living or recently living organisms. Examples of biomass fuel include wood, charcoal, hydrogen gas, alcohol (e.g., ethanol), organic oils (e.g., palm oil, rapseed, jathorpa), manure, grass cuttings, and biodiesel. Sources of biological materials used in biomass fuel include, but are not limited to trees (e.g., poplar, pine, willow, oak, maple, eucalyptus, oil palm), miscanthus, switchgrass, hemp, corn, cassava, sorghum, sugarcane, sugar beet, soybean, sunflower, wheat, rapeseed, jathorpa, salicornia, mahua, mustard, flax, field pennycress, pongamia pinnata, and algae.

“Hydrocarbon isomerization” refers to a reaction which converts an n-alkane into a branched alkane and/or a cycloalkane, such as cylcohexane or methylcyclopentane, or which increases the amount of branching in an alkane-containing molecule. Hydrocarbon isomerization does not involve cracking and, as such, is not part of an upgrading process, but hydrocarbon isomerization can occur during reforming. In hydrocarbon isomerization, old C—C bonds are broken and new C—C bonds are formed, but the total number of carbon atoms in a molecule remains the same.

Hydrocarbon isomerization is also distinct from dehydrosulfurization, which removes covalently bound sulfur from a feedstock; from hydrodeoxygenation, which removes covalently bound oxygen from a feedstock; and from hydrotreating, which can remove covalently bound sulfur, oxygen, or nitrogen from a feedstock.

In some instances, the alditol is not completely hydrodeoxygenated prior to isomerization, resulting in a complex mixture of branched, intermediately deoxygenated products such as 3-hexanone, 2-hexanone, 1-hexanol, hexanoic acid, and in cyclic products such as cyclic ethers, for example saturated furans and pyrans. This isomerization is also considered a hydrocarbon isomerization, as used herein.

“Hydrogenolysis” refers to cleavage of a carbon-carbon or carbon-heteroatom single bond by hydrogen, typically when the reaction is catalyzed. The heteroatom can be, for example, oxygen, nitrogen, or sulfur. A related reaction is hydrogenation, where hydrogen is added to the molecule, without altering atom connectivity.

“Hydrodeoxygenation” refers to a process where the C—O bond in oxygenated compounds is cleaved by hydrogen gas. Hydrodeoxygenation catalysts can include, for example, porous nickel supported on silica, Pt/Nb₂O₅, nickel-molybdenum (NiMo), cobalt-molybdenum on gamma alumina, zeolites (e.g., ZSM-5), palladium on carbon (Pd/C), platinum on carbon (Pt/C), and platinum on alumina.

“Hydrodesulfurization” (HDS) refers to a catalytic chemical process to remove sulfur from natural gas and refined petroleum products, such as gasoline, jet fuel, kerosene, diesel fuel, and fuel oils. Mercaptan-containing organics are catalytically hydrogenated to remove sulfur and generate H₂S as a byproduct. Industrial hydrodesulfurization captures and removes the resulting H₂S, typically converting it into elemental sulfur or sulfuric acid. Most HDS catalysts are sulfides, such as ReS₂, MoS₂, optionally containing small amount of another metal. HDS catalysts can also comprise an alumina base impregnated with cobalt and molybdenum (CoMo), or a combination of nickel and molybdenum (NiMo) with CoMo. The latter catalyst is especially useful for stocks with a high level of chemically bound nitrogen.

“Hydrotreating” refers hydrogenolysis wherein carbon-heteroatom bonds, for example C—N and C—S bonds, are broken. As such, the term “hydrotreating” encompasses the meaning of hydrodesulfurization.

“Naphtha” refers a flammable liquid mixture of hydrocarbons, for example a distillation product from petroleum or coal tar boiling. Naphtha broadly covers the lightest and most volatile fraction of the liquid hydrocarbons in petroleum. Full range naphtha is a fraction of hydrocarbons in petroleum boiling between about 30° C. and about 250° C., consisting essentially of a complex mixture of hydrocarbon molecules having between about 5 and about 12 carbon atoms. Naphtha typically constitutes about 15% to about 30% of crude oil by weight. Light naphtha is the fraction boiling between about 30° C. and about 90° C., consisting essentially of molecules with 5 to 6 carbon atoms. Heavy naphtha boils between about 90° C. and about 250° C., consisting essentially of molecules with 6 to 12 carbons. Naphtha can be used as a feedstock to produce high-octane gasoline via reforming, such as catalytic reforming, to produce olefins in steam crackers, and as a solvent.

“Octane rating” or “octane number” refers to a measure of detonation resistance in gasoline, indicating a fuel's tendency to burn controllably. Octane rating is not a measure of a fuel's heat content. Octane number is measured in a test engine, and is defined by comparing a mixture of 2,2,4-trimethylpentane (isooctane) and n-heptane with the same anti-knocking capacity as the tested fuel: The percentage, by volume, of 2,2,4-trimethylpentane in that mixture is the octane number of the fuel. For example, gasoline with the same knocking characteristics as a 90:10 mixture of isooctane/n-heptane has an octane rating of 90. Because some fuels are more knock-resistant than isooctane, octane numbers higher than 100 are allowed.

“Olefin” refers to a chemical comprising at least one carbon-carbon double bond, particularly chemicals made from oil or natural gas feedstocks. Olefins are commonly used to manufacture plastics and gasoline. Examples of olefins include, but are not limited to, ethylene (acetylene), propylene (propene), butene, pentene, hexane, heptene, octene, nonene, decene, undecene, dodecene, eicosene, and butadiene

“Oxygenate” refers to a chemical compound comprising oxygen, and usually refers to oxygenated fuels. Oxygenates can be added to gasoline to increase fuel burning efficiency, for example, by reducing carbon monoxide during combustion. Oxygenates include, but are not limited to:
alcohols, for example methanol (MeOH), ethanol (EtOH), isopropyl alcohol (IPA), n-butanol (BuOH), gasoline grade t-butanol (GTBA), pentanol, hexanol, cyclopentanol, cyclohexanol, and 2-methylcyclopentanol;
polyols such as alditols;
ethers, such as methyl tert-butyl ether (MTBE), tertiary amyl methyl ether (TAME), tertiary hexyl methyl ether (THEME), ethyl tertiary butyl ether (ETBE), tertiary amyl ethyl ether (TAEE), disopropyl ether (DIPE), furan, tetrahydrofuran, dihydrofuran, 2-furanomethanol, 2-methyl-tetrahydrofuran, 2,5-dimethyl-tetrahydrofuran, 2-ethyltetrahydrofuran, 2-methylfuran, 2,5-dimethylfuran, 2-ethylfuran, 3-hydroxytetrahydrofuran, tetrahydro-3-furanol, tetrahydrorfurfuryl alcohol, and 1-(2-furyl)ethanol;
ketones, such as hydroxyketones, cyclic ketones, acetone, propanone, butanone, pentanone, hexanone, 2-methyl-cyclopentanone, butanedione, pentanedione, diketo-hexane, 5-hydroxymethyl-2(5H)furanone, dihydro-5-(hydroxylmethyl)-2-(3H)-furanone, and dihydro-5-(hydroxymethyl)-2(3H)-furanone;
aldehydes, such as acetaldehyde, propionaldehyde, butyraldehyde, pentanal, hexanal, furfural, hydroxymethylfurfural, and hydroxymethyltetrahydrofurfural; and
carboxylic acids, such as formic acid, acetic acid, propionic acid, butanoic acid, pentanoic acid, hexanoic acid, lactic acid, and tetrahydro-2-furanoic acid.

“Paraffin” or “alkane” refers to a saturated hydrocarbon with straight or branched chains consisting of carbon and hydrogen and having the general formula CₙH₂ₙ₊₂. Paraffin generally has from about 5 to about 40 carbon atoms per molecule.

“Polyol” or “polyhydric alcohol” refers to an alcohol containing multiple hydroxyl groups. A molecule with two hydroxyls is a diol, with three hydroxyls is a triol, with four hydroxyls is a tetrol, and so on. Polyols can include, but are not limited to, sugar alcohols, such as alditol, maltitol, sorbitol, xylitol and isomalt; sugars, such as aldose, glucose, fructose, lactose, maltose, xylose, and sucrose; pentaerythritol, ethylene glycol, glycerin, castor oil; and polyether diols, such as polyethylene glycol, polypropylene glycol, and poly(tetramethylene ether) glycol.

“Reform”, “reforming”, or “reformation” as used herein refers hydrocarbon isomerization; that is, to a reaction which converts an n-alkane to a branched alkane and/or cycloalkane or increases the amount of branching of an alkane-containing molecule. For example, reforming is used to convert naphtha with a low octane rating into high-octane liquid products, referred to as “reformates”. These liquid products are components of high-octane gasoline. Overall, reformate contains more complex hydrocarbons with higher octane values than hydrocarbons in the feedstock. Byproducts include mostly hydrogen and small amounts of methane, ethane, propane and butane.

In catalytic reforming, platinum and rhenium can be catalytic sites for dehydrogenation, and chlorinated alumina or tungstated zirconia provides acid sites for isomerization, cyclization and hydrocracking. Coke deposition (carbonaceous build-up on the catalyst) and chloride loss result in decreased catalytic activity. The catalyst can be periodically regenerated or restored by in situ high-temperature oxidation of the coke, then chlorination, for example, once every 6 to 24 months up to about 4 times.

“Tungstated zirconia” or “WZ” refers to a chemical composition comprising tungsten oxide (WO₃) and zirconia (ZrO₂). Tungstated zirconia can be promoted with a noble metal, for example, Pt, Pd, Re, Ru, Rh, or combinations thereof. For example, platinum-promoted tungstated zirconia is denoted as “Pt/WO₃—ZrO₂” or “Pt/WZ”. Pt is preferred because of the relatively low occurrence of carbon-carbon bond cleavage when this metal is used. Highly acidic supports/catalysts can be used for the isomerization/reforming reaction. The components of the WO₃/ZrO₂ used herein work together to make a very highly acidic solid catalyst.

“Upgrade” or “upgrading” as used herein refers to a process which involves cracking and/or removal unwanted impurities from a hydrocarbon fuel, such as by distillation, filtration, hydrotreating, hydrodesulfurization or hydrodeoxygenation. Upgrading can comprise vacuum distilling to separate lighter fractions, which leaves a residue with molecular weights of at least 400; de-asphalting the residue to remove the highest molecular weight alicyclic compounds, which precipitate as black/brown asphaltenes when the mixture is dissolved in C₃-C₇ alkanes; and hydrotreating to remove sulfur and nitrogen. As used herein, the terms “upgrade” and “upgrading” do not include hydrocarbon isomerization which transforms an n-alkane into a branched alkane and/or cycloalkane or which increases the amount of branching in an alkane-containing molecule.

The present invention is exemplified with respect to hydrodeoxygenation and hydrocarbon isomerization of Alditols. However, this process is exemplary only, and the invention can be broadly applied to reforming and upgrading any fuel feedstock comprising oxygenates. The following examples are intended to be illustrative only, and not unduly limit the scope of the appended claims.

### Example 1

### Catalyst Preparation

ZrOCl₂ (500 g) was dissolved in 4 L of deionized (DI) water and 220 mL of concentrated ammonium hydroxide (NH₄OH) was added. The mixture was stirred for 1 hour, filtered and washed with 8 L of DI water. The solid was dried at 120° C. under vacuum, then ground to 35-100 mesh (about 0.10-0.50 mm average particle size). The catalyst was mixed to incipient wetness with 15 wt % aqueous ammonium metatungstate ((NH₄)₆(W₁₂O₄₁)).

The mixture was dried and calcined at 700° C. for 3 hours. This calcination protocol was selected to increase the tetragonal zirconia content of the catalyst. Twenty grams of the calcinated mixture were loaded with 0.397 g of Pt(NH₃)₄(NO₃)₂ dissolved in enough DI water to make sufficient solution to bring the catalyst to incipient wetness. The damp mixture was then calcined a second time at 300° C. for at least 12 hours to produce the final catalyst. The catalyst was approximately 18 wt % WO₃, 81 wt % ZrO₂, and 1 wt % Pt based on the total weight of the catalyst. In other words, the ratio of Pt/WO₃/ZrO₂ was about 1:18:81.

Catalyst synthesis correlates with catalyst reactivity. Without wishing to be bound by theory, it is believed that Pt serves a dual role in this reaction, providing hydrogenation centers needed for hydrogenolysis and keeping the catalyst active by supplying spillover hydrogen to the surface, which delays deactivation.

### Example 2

### Hydrodeoxygenation and Isomerization

Exemplary reactions demonstrating the invention were carried out in a ¾-inch diameter down-flow reactor. Ten milliliters of catalyst (as described in Example 1) were diluted with 2 mL of alundum (fused alumina) to make the catalyst bed. The catalyst was reduced at 275° C. in the presence of excess hydrogen (about 100 sccm) for an hour before use.

For the reaction, 200 sccm of hydrogen, 10 mL/hour n-hexadecane, and 10 mL/hour of 40 weight % aqueous sorbitol were delivered to the reactor. The pressure was maintained at 850 psig throughout the reaction. The temperature was ramped up during the run from 150° C. to 275° C. At the highest temperature, about 42% of the oxygenate feed was converted into non-aqueous products. As discussed previously, the product stream was complex. n-Hexane, cyclohexane and methylcyclopentane were some of the hydrocarbons produced. We also observed a complex mixture of intermediately deoxygenated products such as 3-hexanone, 2-hexanone, 1-hexanol, hexanoic acid, and cyclic ethers, for example saturated furans, pyrans, and their isomers. With further or more severe processing, these products would have also been deoxygenated.

The following references are incorporated by reference in their entirety:
U.S. Pat. No. 4,338,472;
U.S. Pat. No. 4,366,332;
US2008216391;
US20090255171;
Busto et al., “Pt—Pd/WO₃—ZrO₂ catalysts for isomerization-cracking of long paraffins”, Applied Catalysis A: General, 2008, 347(2): 117-125;
Gong et al. “Solvent Effect on Selective Dehydroxylation of Glycerol to 1,3-Propanediol over Pt/WO₃/ZrO₂”, Chinese Journal of Catalysis, 2009, 30(12): 1189-1191;
Hattori et al., “Participation of the Protonic Acid Sites Originating from Molecular Hydrogen in Alkane Skeletal Isomerization Catalyzed by Pt/WO₃—ZrO₂”, Proceedings of 13^{th} Saudi-Japanese Catalyst Symposium, December 2003, 124-133;
Hong et al., “Hydrodeoxygenation and coupling of aqueous phenolics over bifunctional zeolite-supported metal catalysts.” Chem Commun, 2010, 46: 1038-1040;
Khurshid & Al-Khattaf, “n-Heptane isomerization over Pt/WO₃—ZrO₂: A kinetic study”, Applied Catalysis A: General, 2009, 368(1-2): 56-64;
Kurosaka et al., “Production of 1,3-propanediol by hydrogenolysis of glycerol catalyzed by Pt/WO₃/ZrO₂”, Catalysis Communications, 2008, 9(6): 1360-1363;
Lukinskas et al., “Role of promoters on tungstated zironia catalysts”, Topics in Catalysis, 2003, 23(1-4): 163;
Song et al., “n-Hexane Isomerization by Pt/WO₃—ZrO₂ Using Hydrothermally Synthesized Hydrous Zirconia as Support”, Chinese J. of Catal., 2008, 29(12): 1196-1198;
Triwahyono et al., “Molecular Hydrogen Originated Protonic Acid Site on Pt/WO₃—ZrO₂”, Journal of Natural Gas Chemistry, 2007, 16(3): 252-257; and
Yori et al., “n-Butane isomerization of Pt/WO₃—ZrO₂: effect of the Pt incorporation”, Applied Catalysis A: General, 1999, 181(1): 5-14.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

What is claimed is:

1. A method for hydrocarbon processing, comprising:
providing a fluid hydrocarbon feedstock comprising an oxygenate;
contacting the feedstock with a catalyst comprising Pt-loaded WO₃/ZrO₂ in the presence of hydrogen; and
reacting the feedstock, hydrogen and catalyst at a temperature and a pressure for a period of time sufficient to allow both hydrodeoxygenation and hydrocarbon isomerization of said oxygenate.

2. The method of claim 1, wherein the fluid hydrocarbon feedstock comprises an aqueous oxygenate stream and a fluid hydrocarbon stream provided substantially simultaneously.

3. The method of claim 1, wherein ratio of WO₃ to ZrO₂ is from 1:9 to 2:8.

4. The method of claim 1, wherein the catalyst was formed at a calcination temperature of 400° C. to 800° C. for 2 hours to 4 hours, sufficient to increase the tetragonoal zirconia content of the catalyst.

5. The method of claim 1, wherein the weight percent of Pt in the catalyst is 0.01 wt % to 10 wt % of the weight of the catalyst.

6. The method of claim 5, wherein the weight percent of Pt in the catalyst is 1 wt % to 5 wt % of the weight of the catalyst.

7. The method of claim 1, wherein the catalyst is substantially free of sulfide.

8. The method of claim 1, wherein the catalyst has an average particle size of 10 μm to 10 mm.

9. The method of claim 8, wherein the catalyst has an average particle size of 100 μm to 500 μm.

10. The method of claim 1, wherein the catalyst is porous.

11. The method of claim 1, further comprising: separating a product from the feedstock, hydrogen and catalyst.

12. The method of claim 1, wherein the feedstock is a biofuel.

13. The method of claim 1, wherein the oxygenate comprises at least one alditol.

14. The method of claim 13, wherein the alditol is selected from the group consisting of erythritol, threitol, arabitol, xylitol, ribitol, sorbitol, iditol, dulcitol, mannitol and mixtures thereof.

15. The method of claim 1, wherein the temperature is from 100° C. to 300° C.

16. The method of claim 15, wherein the temperature is from 150° C. to 250° C.

17. The method of claim 1, wherein the pressure is 800 psig to 2500 psig.

18. The method of claim 1, wherein the period of time is from 15 minutes to 5 hours.

19. A method for hydrodeoxygenation and isomerization of an alditol, comprising:
providing substantially simultaneously a fluid hydrocarbon stream and an aqueous stream comprising at least one alditol;
contacting the streams with a catalyst comprising Pt-loaded WO₃/ZrO₂ in the presence of hydrogen; and
reacting the streams, hydrogen and catalyst from 100° C. to 300° C. at 800 psig to 2500 psig for 15 minutes to 5 hours sufficient to allow both hydrodeoxygenation and isomerization of the alditol.

20. The method of claim 15, wherein the alditol is selected from the group consisting of erythritol, threitol, arabitol, xylitol, ribitol, sorbitol, iditol, dulcitol, mannitol and mixtures thereof.
